SEQUENCE LISTING (14 sequences)

SEQ ID NO: 1 - RNA, 22 nt, unidentified
  source 1..22: mol_type = transcribed RNA; note = human
  misc_feature 1..22: note = hsa-miR-205-5p
tccttcattccaccggagtctg

SEQ ID NO: 2 - RNA, 22 nt, unidentified
  source 1..22: mol_type = transcribed RNA; note = human
  misc_feature 1..22: note = hsa-miR-223-5p
cgtgtatttgacaagctgagtt

SEQ ID NO: 3 - RNA, 22 nt, unidentified
  source 1..22: mol_type = transcribed RNA; note = human
  misc_feature 1..22: note = hsa-miR-29c-3p
tagcaccatttgaaatcggtta

SEQ ID NO: 4 - RNA, 23 nt, unidentified
  source 1..23: mol_type = transcribed RNA; note = human
  misc_feature 1..23: note = hsa-miR-324-3p
cccactgccccaggtgctgctgg

SEQ ID NO: 5 - RNA, 22 nt, unidentified
  source 1..22: mol_type = transcribed RNA; note = human
  misc_feature 1..22: note = hsa-miR-34a-5p
tggcagtgtcttagctggttgt

SEQ ID NO: 6 - RNA, 22 nt, unidentified
  source 1..22: mol_type = transcribed RNA; note = human
  misc_feature 1..22: note = hsa-miR-483-5p
aagacgggaggaaagaagggag

SEQ ID NO: 7 - RNA, 22 nt, unidentified
  source 1..22: mol_type = transcribed RNA; note = human
  misc_feature 1..22: note = hsa-miR-885-5p
tccattacactaccctgcctct

SEQ ID NO: 8 - RNA, 22 nt, unidentified
  source 1..22: mol_type = transcribed RNA; note = human
  misc_feature 1..22: note = hsa-miR-486-5p
tcctgtactgagctgccccgag

SEQ ID NO: 9 - RNA, 21 nt, unidentified
  source 1..21: mol_type = transcribed RNA; note = human
  misc_feature 1..21: note = hsa-miR-106b-5p
taaagtgctgacagtgcagat

SEQ ID NO: 10 - RNA, 24 nt, unidentified
  source 1..24: mol_type = transcribed RNA; note = human
  misc_feature 1..24: note = hsa-miR-125a-5p
tccctgagaccctttaacctgtga

SEQ ID NO: 11 - RNA, 22 nt, unidentified
  source 1..22: mol_type = transcribed RNA; note = human
  misc_feature 1..22: note = hsa-miR-190a-5p
tgatatgtttgatatattaggt

SEQ ID NO: 12 - RNA, 22 nt, unidentified
  source 1..22: mol_type = transcribed RNA; note = human
  misc_feature 1..22: note = hsa-miR-30e-5p
tgtaaacatccttgactggaag

SEQ ID NO: 13 - RNA, 24 nt, unidentified
  source 1..24: mol_type = transcribed RNA; note = human
  misc_feature 1..24: note = hsa-miR-3613-3p
acaaaaaaaaaagcccaacccttc

SEQ ID NO: 14 - RNA, 22 nt, unidentified
  source 1..22: mol_type = transcribed RNA; note = human
  misc_feature 1..22: note = hsa-miR-3613-5p
tgttgtacttttttttttgttc

(19) United States
(12) Patent Application Publication
(10) Pub. No.: US 2025/0376735 A1
(43) Pub. Date: Dec. 11, 2025

(54) ANALYSIS METHOD, MARKER, AND KIT

(71) Applicant: KABUSHIKI KAISHA TOSHIBA, Kawasaki-shi (JP)

(72) Inventors: Yoshitake SANO, Adachi (JP); Sunao YOTSUTSUJI, Kawasaki (JP); Tomomi ANDO, Ota (JP); Masaaki TAKADA, Kashiwa (JP); Hisashi YAMADA, Kawasaki (JP); Mitsuko ISHIHARA, Setagaya (JP)

(73) Assignee: KABUSHIKI KAISHA TOSHIBA, Kawasaki-shi (JP)

(21) Appl. No.: 19/312,732

(22) Filed: Aug. 28, 2025

Related U.S. Application Data
(63) Continuation of application No. PCT/JP2024/030544, filed on Aug. 27, 2024.

(30) Foreign Application Priority Data
Nov. 15, 2023 (JP) 2023-194102

(51) Int. Cl. C12Q 1/6886

(52) U.S. Cl. CPC C12Q 1/6886; C12Q 2600/158; C12Q 2600/178

(57) ABSTRACT

According to one embodiment, an analysis method for determining a presence or absence of pancreatic cancer in a test subject, comprising quantifying corrective miRNA and target miRNAs in a sample derived from the test subject, wherein the target miRNAs are three or more selected from a group consisting of hsa-miR-205-5p, hsa-miR-223-5p, hsa-miR-29c-3p, hsa-miR-324-3p, hsa-miR-34a-5p, hsa-miR-483-5p and hsa-miR-885-5p.

### CROSS-REFERENCE TO RELATED APPLICATIONS

This application is a Continuation Application of PCT Application No. PCT/JP2024/030544, filed Aug. 27, 2024 and based upon and claiming the benefit of priority from Japanese Patent Application No. 2023-194102, filed Nov. 15, 2023, the entire contents of all of which are incorporated herein by reference.

### REFERENCE TO A SEQUENCE LISTING

A Sequence Listing, submitted as an XML file and compliant with WIPO Standard ST. 26, forms part of the present application. The Sequence Listing is identified as follows: File name “559526US. xml,” created on Aug. 27, 2025, with a size of 20,965 bytes.

### FIELD

Embodiments described herein relate generally to analytical methods, marker and kit.

### BACKGROUND

Recently, the relationship between microRNAs (miRNAs) and diseases has been attracting attention. It has been reported that miRNAs have the function of regulating gene expression, and their types and expression levels are altered from the initial stage in various diseases. In other words, the amount of certain miRNAs is increased or decreased in patients with a certain disease compared to healthy individuals. Therefore, examination of the amount of such miRNAs in samples collected from a subject is a means of knowing whether the patient is suffering from the disease or not.

### BRIEF DESCRIPTION OF THE DRAWINGS

FIG. 1 is a flowchart showing an example of the analysis method of the first embodiment, wherein (a) is an analysis method including a quantification process of corrective miRNA and target miRNAs wherein the target miRNAs are three or more selected from a group consisting of hsa-miR-205-5p, hsa-miR-223-5p, hsa-miR-29c-3p, hsa-miR-324-3p, hsa-miR-34a-5p, hsa-miR-483-5p and hsa-miR-885-5p, (b) is an example of an analysis method including a determination step to determine whether or not the subject is affected by pancreatic cancer, (c) is an example of an analysis method including a determination step to determine whether or not there is prognosis or recurrence, and (d) is an example of an analysis method including a selection step to select a type of treatment or drug to be applied to the subject.

FIG. 2 is a flowchart of an example of an algorithm construction step which may be included in the determination step of the analysis method of the first embodiment.

FIG. 3 is a flowchart of an example of calculation and comparison steps which may be included in the determination step of the analysis method of the first embodiment.

### DETAILED DESCRIPTION

In general, according to one embodiment, an analysis method for determining a presence or absence of pancreatic cancer in a test subject, comprising: quantifying corrective miRNA and target miRNAs in a sample derived from the test subject, wherein the target miRNAs are three or more selected from a group consisting of hsa-miR-205-5p, hsa-miR-223-5p, hsa-miR-29c-3p, hsa-miR-324-3p, hsa-miR-34a-5p, hsa-miR-483-5p and hsa-miR-885-5p.

Hereinafter, the analysis methods, markers and kits of the embodiments will be described with reference to the drawings.

### First embodiment

### (Analysis Method)

The analysis method of the first embodiment is a method for determining the presence or absence of pancreatic cancer in a test subject, including quantifying corrective miRNA and target miRNAs, wherein the target miRNAs are three or more selected from a group consisting of hsa-miR-205-5p, hsa-miR-223-5p, hsa-miR-29c-3p, hsa-miR-324-3p, hsa-miR-34a-5p, hsa-miR-483-5p, and hsa-miR-885-5p in a sample derived from the subject.

The above-mentioned miRNA groups, which consists 7 types of miRNAs, may be referred to as “target miRNA groups” in the following description.

The individual miRNA of the target miRNA group may be referred to as “target miRNA”. Each target miRNA contains each nucleotide sequences of SEQ ID No. 1 to 7 listed in Table 1 below. In the present application, the letter T of the RNA sequence in the sequence listing means U.

TABLE 1

| SEQ | | |
|---|---|---|
| ID | | |
| NO. | miRNA name | Base sequence |
| 1 | hsa-miR-205-5p | UCCUUCAUUCCACCGGAGUCUG |
| 2 | hsa-miR-223-5p | CGUGUAUUUGACAAGCUGAGUU |
| 3 | hsa-miR-29c-3p | UAGCACCAUUUGAAAUCGGUUA |
| 4 | hsa-miR-324-3p | CCCACUGCCCCAGGUGCUGCUGG |
| 5 | hsa-miR-34a-5p | UGGCAGUGUCUUAGCUGGUUGU |
| 6 | hsa-miR-483-5p | AAGACGGGAGGAAAGAAGGGAG |
| 7 | hsa-miR-885-5p | UCCAUUACACUACCCUGCCUCU |

The target miRNAs to be quantified in the present embodiment may be a combination of three or more target miRNAs, for example, a combination of three target miRNAs, four target miRNAs, or five target miRNAs. However, it is preferable that the combination has been previously investigated and confirmed for sensitivity and specificity in identifying pancreatic cancer incidence under specific conditions (e.g., different types of analysis target, sample, detection method and quantification method). Specifically, the combinations shown in Table 2-1 to Table 2-3 below (i.e., combination No. 1 to 85) are preferred. As will be explained in the examples below, among the combination No. 1 to 85, it was confirmed that the combination No. 1 to 3, 31 to 40, and 65 to 73 showed particularly excellent performance in identifying pancreatic cancer. Therefore, it is even more desirable that the target miRNAs to be quantified are combinations of target miRNAs of combination No. 1 to 3, 31 to 40, and 65 to 73.

TABLE 2

| Combination | | | | | |
|---|---|---|---|---|---|
| No. | miRNA 1 | miRNA 2 | miRNA 3 | miRNA 4 | miRNA 5 |
| 1 | 205-5p | 324-3p | 483-5p | | |
| 2 | 205-5p | 223-5p | 483-5p | | |
| 3 | 205-5p | 29c-3p | 483-5p | | |
| 4 | 29c-3p | 324-3p | 483-5p | | |
| 5 | 223-5p | 324-3p | 483-5p | | |
| 6 | 324-3p | 34a-5p | 483-5p | | |
| 7 | 223-5p | 29c-3p | 483-5p | | |
| 8 | 29c-3p | 483-5p | 885-5p | | |
| 9 | 29c-3p | 34a-5p | 483-5p | | |
| 10 | 205-5p | 29c-3p | 885-5p | | |
| 11 | 324-3p | 483-5p | 885-5p | | |
| 12 | 29c-3p | 324-3p | 885-5p | | |
| 13 | 29c-3p | 324-3p | 34a-5p | | |
| 14 | 205-5p | 324-3p | 885-5p | | |
| 15 | 223-5p | 483-5p | 885-5p | | |
| 16 | 205-5p | 223-5p | 885-5p | | |
| 17 | 223-5p | 29c-3p | 885-5p | | |
| 18 | 205-5p | 29c-3p | 34a-5p | | |
| 19 | 205-5p | 483-5p | 885-5p | | |
| 20 | 205-5p | 324-3p | 34a-5p | | |
| 21 | 223-5p | 324-3p | 885-5p | | |
| 22 | 29c-3p | 34a-5p | 885-5p | | |
| 23 | 223-5p | 29c-3p | 34a-5p | | |
| 24 | 223-5p | 324-3p | 34a-5p | | |
| 25 | 205-5p | 223-5p | 34a-5p | | |
| 26 | 223-5p | 34a-5p | 483-5p | | |
| 27 | 324-3p | 34a-5p | 885-5p | | |
| 28 | 205-5p | 34a-5p | 483-5p | | |
| 29 | 223-5p | 34a-5p | 885-5p | | |
| 30 | 205-5p | 34a-5p | 885-5p | | |
| 31 | 29c-3p | 324-3p | 34a-5p | 483-5p | |
| 32 | 205-5p | 29c-3p | 324-3p | 483-5p | |
| 33 | 205-5p | 324-3p | 34a-5p | 483-5p | |
| 34 | 205-5p | 29c-3p | 34a-5p | 483-5p | |
| 35 | 205-5p | 223-5p | 324-3p | 483-5p | |
| 36 | 205-5p | 223-5p | 29c-3p | 483-5p | |
| 37 | 29c-3p | 324-3p | 483-5p | 885-5p | |
| 38 | 205-5p | 29c-3p | 483-5p | 885-5p | |
| 39 | 223-5p | 324-3p | 34a-5p | 483-5p | |
| 40 | 205-5p | 324-3p | 483-5p | 885-5p | |
| 41 | 205-5p | 223-5p | 483-5p | 885-5p | |
| 42 | 223-5p | 29c-3p | 324-3p | 483-5p | |
| 43 | 223-5p | 29c-3p | 483-5p | 885-5p | |
| 44 | 223-5p | 29c-3p | 34a-5p | 483-5p | |
| 45 | 223-5p | 324-3p | 483-5p | 885-5p | |
| 46 | 205-5p | 223-5p | 34a-5p | 483-5p | |
| 47 | 205-5p | 29c-3p | 324-3p | 885-5p | |
| 48 | 205-5p | 29c-3p | 324-3p | 34a-5p | |
| 49 | 29c-3p | 324-3p | 34a-5p | 885-5p | |
| 50 | 205-5p | 29c-3p | 34a-5p | 885-5p | |
| 51 | 223-5p | 29c-3p | 324-3p | 34a-5p | |
| 52 | 205-5p | 223-5p | 29c-3p | 885-5p | |
| 53 | 205-5p | 223-5p | 324-3p | 885-5p | |
| 54 | 223-5p | 29c-3p | 324-3p | 885-5p | |
| 55 | 205-5p | 223-5p | 29c-3p | 34a-5p | |
| 56 | 324-3p | 34a-5p | 483-5p | 885-5p | |
| 57 | 205-5p | 324-3p | 34a-5p | 885-5p | |
| 58 | 223-5p | 29c-3p | 34a-5p | 885-5p | |
| 59 | 205-5p | 223-5p | 324-3p | 34a-5p | |
| 60 | 29c-3p | 34a-5p | 483-5p | 885-5p | |
| 61 | 223-5p | 324-3p | 34a-5p | 885-5p | |
| 62 | 205-5p | 223-5p | 34a-5p | 885-5p | |
| 63 | 223-5p | 34a-5p | 483-5p | 885-5p | |
| 64 | 205-5p | 34a-5p | 483-5p | 885-5p | |
| 65 | 205-5p | 29c-3p | 324-3p | 34a-5p | 483-5p |
| 66 | 205-5p | 29c-3p | 324-3p | 483-5p | 885-5p |
| 67 | 205-5p | 223-5p | 324-3p | 34a-5p | 483-5p |
| 68 | 205-5p | 223-5p | 29c-3p | 34a-5p | 483-5p |
| 69 | 223-5p | 29c-3p | 324-3p | 34a-5p | 483-5p |
| 70 | 205-5p | 223-5p | 29c-3p | 324-3p | 483-5p |
| 71 | 205-5p | 223-5p | 29c-3p | 483-5p | 885-5p |
| 72 | 205-5p | 223-5p | 324-3p | 483-5p | 885-5p |
| 73 | 223-5p | 29c-3p | 324-3p | 483-5p | 885-5p |
| 74 | 29c-3p | 324-3p | 34a-5p | 483-5p | 885-5p |
| 75 | 205-5p | 324-3p | 34a-5p | 483-5p | 885-5p |
| 76 | 205-5p | 29c-3p | 34a-5p | 483-5p | 885-5p |
| 77 | 205-5p | 29c-3p | 324-3p | 34a-5p | 885-5p |
| 78 | 205-5p | 223-5p | 29c-3p | 324-3p | 34a-5p |
| 79 | 223-5p | 324-3p | 34a-5p | 483-5p | 885-5p |
| 80 | 205-5p | 223-5p | 29c-3p | 324-3p | 885-5p |
| 81 | 205-5p | 223-5p | 29c-3p | 34a-5p | 885-5p |
| 82 | 223-5p | 29c-3p | 34a-5p | 483-5p | 885-5p |
| 83 | 223-5p | 29c-3p | 324-3p | 34a-5p | 885-5p |
| 84 | 205-5p | 223-5p | 34a-5p | 483-5p | 885-5p |
| 85 | 205-5p | 223-5p | 324-3p | 34a-5p | 885-5p |

Corrective miRNA is a miRNA which has been previously confirmed to be commonly present at a certain level in samples derived from pancreatic cancer patients and samples derived from individuals other than pancreatic cancer patients. For example, hsa-miR-486-5p (SEQ ID No. 8: UCCUGUACUGAGCUGCCCCGAG), which is known to be commonly expressed in pancreatic cancer patients, healthy individuals, and various cancer patients other than pancreatic cancer, will be used as a corrective miRNA.

The test subject is the animal provided for the analysis using the present method, i.e., the animal providing the sample. The test subject may be an animal with some disease or a healthy animal. For example, the test subject may be an animal that may have cancer or has had cancer in the past, etc. In particular, it may be an animal that may have pancreatic cancer or has had pancreatic cancer in the past, etc. It is preferred that the test subject be a human.

Alternatively, the test subject may be another animal. Other animals are, for example, mammals, including primates such as monkeys, rodents such as mice, rats or guinea pigs, companion animals such as dogs, cats or rabbits, domestic animals such as horses, cows or pigs, or animals belonging to exhibition animals, etc. If the animal is other than human, the target miRNAs are a corresponding miRNA present in the animal.

Subject-derived samples include samples taken from the subject or samples which have been appropriately processed. Samples are preferably serum or plasma. The sample may be other body fluids, such as blood, leukocyte interstitial fluid, urine, stool, sweat, saliva, oral mucosa, nasal mucosa, nasal discharge, pharyngeal mucosa, sputum, digestive fluid, gastric fluid, lymph fluid, spinal fluid, tear fluid, breast milk, amniotic fluid, semen or vaginal fluid, or the like. Alternatively, the sample may be tissue or cells, etc., and may be tissue or cells collected from the test subject and cultured, or the supernatant thereof.

In this document, various types of “cancer” include any stage of cancer, such as cancer which remains in the organ of origin, cancer which has spread to surrounding tissues, cancer which has metastasized to lymph nodes, and cancer that has metastasized to distant organs, or the like. In this document, pancreatic cancer refers to a malignant tumor (neoplasm) which forms in the pancreatic tissue. For example, pancreatic cancer includes those commonly referred to as “pancreatic cancer”, “pancreatic carcinoma”, “pancreatic duct cancer” or “invasive pancreatic duct cancer”. Also, in this document, various types of cancer include, for example, epithelial tumors, non-epithelial tumors, or tumors composed of both epithelial and non-epithelial tumors.

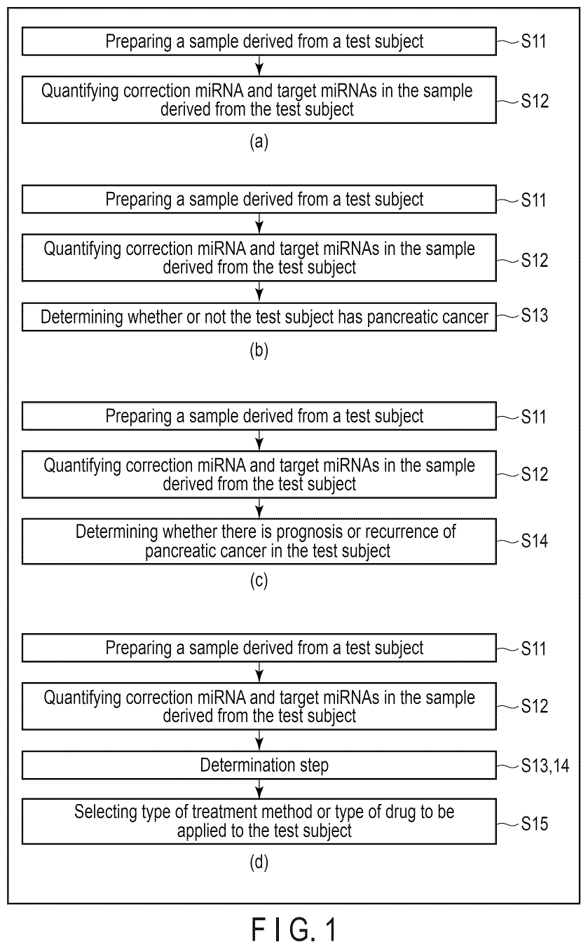

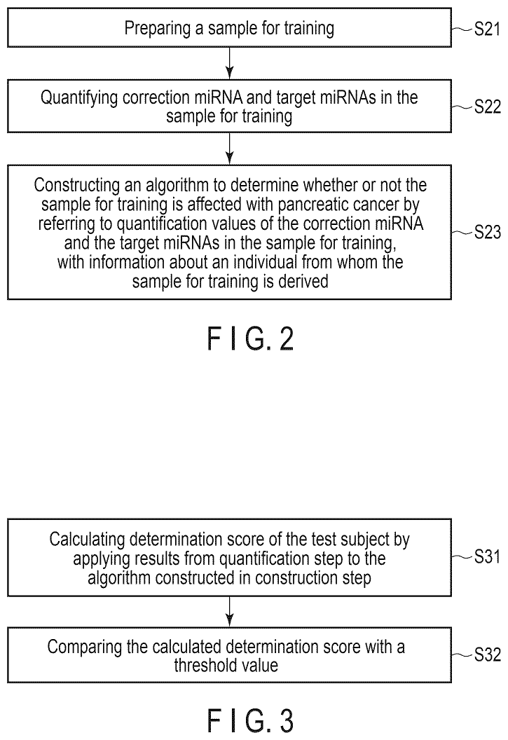

An example of the procedure of the method of the first embodiment will be described below with reference to part (a), (b) and (c) of FIG. 1.

As shown in part (a) of FIG. 1, the analysis method includes, for example: preparing a sample derived from a test subject (preparation step (S11)), and quantifying corrective miRNA and target miRNAs, wherein the target miRNAs are three or more miRNAs of a group of target miRNAs in the sample derived from a test subject (quantification step (S12)).

First, in preparation step (S11), a sample derived from the test subject is prepared. The sample may be collected by a general method appropriate to its type. The sample can be used as it is, or it can be processed so that it does not interfere with the reaction or so that it becomes more suitable for the reaction. The type of processing may be any of the publically known means, such as comminution, homogenization, centrifugation, precipitation, extraction and/or separation.

For example, extraction may be performed using commercially available nucleic acid extraction kits. Nucleic acid extraction kits include, for example, NucleoSpin (registered trademark) miRNA Plasma (Machrei Nagel), Quick-cfRNA Serum & Plasma Kit (Zymo Research), miRNeasy Serum/Plasma Kit (Kiagen), miRVana PARIS isolation kit (Thermo Fisher), PureLink™ Total RNA Blood Kit (Thermo Fisher), Plasma/Serum RNA Purification Kit (Norgen Biotek), microRNA Extractor (registered trademark) SP Kit (Fujifilm Wako Pure Chemicals Corporation), High Pure miRNA Isolation Kit (Sigma-Aldrich), etc. Alternatively, extraction may be performed without using a commercially available kit, for example, by processing the sample with a protein denaturing agent, using an organic solvent or buffer solution, centrifugation, or nucleic acid precipitation. Next, corrective miRNA and the three or more

target miRNAs contained in the sample derived from test subject are quantified (quantification step (S12)). The quantification step (S12) may be performed using a general method for quantifying RNA, especially short-stranded RNA, such as miRNA. For example, a common method includes a step of reverse-transcribing miRNAs, generating cDNA, amplifying the obtained cDNA, and detecting and quantifying the amplified product. It is also common practice to add an artificial sequence to the end of cDNA obtained by reverse transcription in order to facilitate amplification if RNA is short. In addition, the 1-step RT-qPCR method and rolling circle amplification method are known as techniques to directly amplify RNA in a sample without reverse transcription, and to detect and quantify the amplified product. Furthermore, if the concentration of miRNA in the sample is relatively high or if a device capable of high-sensitivity measurement can be used, direct detection of miRNA (or its cDNA) without miRNA amplification may be performed as a common method. For example, a microarray equipped with nucleic acid probes which hybridize and specifically bind to miRNAs can be used as a device capable of direct detection.

For amplification, PCR (including qPCR) or LAMP methods can be used, for example. Detection and quantification may be performed after amplification or over time during amplification. For example, measurement methods using turbidity- or absorbance-based signals, optical or electrochemical signals, or a combination of these methods can be used for detection and quantification. For example, miRNA can be quantified from the intensity or amount of change in the above signals obtained according to the amount of amplified product, or the time it takes for the signal to reach the threshold (rise time), or the number of cycles until the signal rises is measured when the PCR method may be used. The results of next-generation sequencing (NGS) methods, for example, can also be used for detection and quantification. In such cases, relative quantification of miRNAs can be performed from the detection results, such as the number of reads aligned to miRNAs.

The quantitative value of miRNAs may be determined using a calibration curve which represents the relationship between the detection of the above signals and the abundance of miRNAs. The calibration curve can be created based on signal detection results on multiple standard samples containing miRNA at different concentrations. By comparing this calibration curve with the signal detection results obtained for the sample derived from the test subject, the abundance of miRNAs in the sample can be calculated. Abundance of miRNAs in a sample may be calculated, for example, as the number of copies of miRNA per unit volume.

Quantification in the quantification step (S12) may be performed, for example, using commercially available kits. Examples of commercially available kits are TaqMan (registered trademark) Advanced miRNA CDNA Synthesis Kit (Thermo Fisher, catalog No. A28007), TaqMan (registered trademark) Advanced miRNA Assays (Thermo Fisher, catalog No. A25576), TaqMan (registered trademark) Fast Advanced Master Mix (Thermo Fisher, catalog No. 4444964), miRCURY LNA (registered trademark) RT Kit (KIAGEN, catalog No. 339340), miRCURY LNA (registered trademark) miRNA PCR Assays (Kiagen, catalog No. 339306, SYBR (registered trademark) Green qPCR microRNA detection system (Origin Technologies), etc., and they may be used with primers and probes which specifically amplify miRNAs.

For quantification of corrective miRNA and three or more types of target miRNAs, individual reaction systems may be prepared for each type of miRNA, and quantification may be performed by reverse transcription, elongation, amplification and/or detection for each reaction system. Alternatively, multiple types of miRNAs may be detected and quantified in the same reaction system by using, for example, a flow-through chip capable of simultaneously detecting multiple nucleic acids. Or, for example, multiple types of miRNAs may be detected and quantified in the same reaction system by using a probe capable of simultaneously detecting multiple nucleic acids. For example, by using the NGS method, multiple types of miRNAs can be amplified, detected, and quantified in the same reaction system in a massively parallel manner.

Considering that the quantitative values of target miRNAs may differ in the overall activity level of miRNAs from individual to individual and that the yields of extraction and amplification reactions may vary from cell to cell and specimen to specimen in processing specimens, it is preferable to use the quantitative value of the corrective miRNA to normalize the quantitative value of each target miRNA. Specifically, it is preferable to normalize using hsa-miR-486-5p as the corrective miRNA, and the quantification value of each target miRNA is normalized (e.g., converted to a ratio of the corrective miRNA) by the quantification value of the normalizing miRNA in the sample.

The data which relates to the detection of corrective miRNA and three or more target miRNAs obtained in the quantification step (S12) can be used to determine whether or not the test subject has pancreatic cancer. The analysis method of the first embodiment can further include a determination step (S13) of whether or not the subject has pancreatic cancer, which is performed after the quantification step (S12), as shown in part (b) of FIG. 1.

In the determination step (S13), whether or not pancreatic cancer is contained in the sample derived from the subject is determined by comparing certain criteria with the data obtained in the quantification step (S12). In other words, the determination step (S13) can provide information that assists in determining that the test subject is affected with pancreatic cancer. The determination that the subject is “affected” includes the determination that the subject is likely to be affected. Conversely, the determination that is “not affected” includes a determination that the probability of affection is low.

The criteria in the determination step (S13) may be set by referring to the results of the quantification step (S12), or may be a predetermined threshold value (e.g., a value determined from known literature or past findings, etc.). Alternatively, a determination algorithm for calculating a “determination score” representing the incidence of pancreatic cancer may be constructed in advance, and a threshold value which can significantly discriminate the presence or absence of pancreatic cancer among the determination scores calculated using the determination algorithm may be set and used as the criteria.

That is, the determination step (S13) may include a step for constructing a determination algorithm which calculates a determination score. The determination algorithm for determining pancreatic cancer incidence can be constructed based on a quantification data obtained by analyzing a sample derived from an individual known to have pancreatic cancer (i.e., a pancreatic cancer patient) as a sample for training. The quantification analysis of the sample for training may be performed prior to the quantification step (S12) or in parallel with the quantification step (S12).

For example, a step of constructing a determination algorithm may include: (S21) preparing a sample for training; (S22) quantifying the corrective miRNA and the three or more target miRNAs in the prepared sample for training; (S23) constructing a determination algorithm for determining whether the sample for training is affected with pancreatic cancer by referring to a quantification values of the corrective miRNA and the three or more target miRNAs in the sample for training, as well as information about an individual from whom the sample for training is derived. The information on the individual includes information on whether or not the individual has pancreatic cancer, and may also include personal data such as pre-existing medical conditions, sex, BMI, smoking rate, and the like.

Here, “a determination algorithm” means a calculation formula and/or a program which executes a calculation formula, and executes various calculations (e.g., four arithmetic operations and exponential-logarithmic calculations) on the combination of the quantitative values of three or more target miRNAs normalized by the corrective miRNA, and then calculates a determination score which is an index of the morbidity of pancreatic cancer. In other words, the determination score is a numerical index of the probability that the test subject has pancreatic cancer based on the combination of the quantified values of three or more target miRNAs and the quantified values of corrective miRNA, and its calculation method depends on the determination algorithm employed.

The samples for training are derived from individuals who are known to have pancreatic cancer or not, as described above. More specifically, the samples for training are, for example, specimens collected from individuals with pancreatic cancer, specimens collected from individuals other than those with pancreatic cancer, and pancreatic cancer-derived cells. Here, individuals other than pancreatic cancer-affected individuals are, for example, healthy individuals and cancer-affected individuals other than pancreatic cancer. In this document, a healthy individual refers to an individual who does not have at least cancer. A healthy individual is preferably the healthy individual without any disease or abnormality. In addition, a cancer-affected individual other than pancreatic cancer is, for example, an individual afflicted with breast cancer, lung cancer, stomach cancer, and colorectal cancer.

The individual from which the sample for training is derived may be different from the individual to be analyzed in the present method, but it is preferable that it be an individual belonging to the same species, i.e., a human if the test subject is a human. The physical condition or number of individuals, such as age, sex, and height and weight of the control is not limited, but it is more preferable if the physical condition is the same or similar to that of the test subject in the present analysis method.

As a type of determination algorithm, a trained model may be constructed which is trained to enable the determination of pancreatic cancer incidence. Such a model can be created using training data prepared in advance. The training data includes quantitative values of target miRNAs and quantitative values of corrective miRNA in the sample for training.

In order to provide a trained model with higher determination accuracy, it is preferable to have a greater variety and number of individuals from which the samples for training are derived. Therefore, it is preferable to prepare as training data, for example, quantitative analysis results for each specimen collected from a plurality of individuals suffering from cancers other than pancreatic cancer and/or quantitative analysis results for each specimen collected from a plurality of healthy individuals. In addition, such training data includes at least the presence or absence of pancreatic cancer as information about each individual, but may also include other personal data such as, for example, pre-existing medical conditions, sex, BMI, smoking rate, etc.

If the trained model obtained using machine learning methods is, for example, a neural network model, in training the neural network model, the neural network model may be configured such that the result of determining morbidity is obtained as an output when training data is input. Other models, such as linear models, nonlinear models, Bayesian models, support vector machine models, random forest models, boosting models, or the like may be used, not limited to the neural network model as a machine learning model.

The determination algorithm may be constructed such that the determination score is calculated with different weighting for each target miRNA. For example, a determination score may be calculated by constructing an algorithm in which the absolute value of the weighting factor is set high for target miRNAs which have a high relevance with pancreatic cancer and the absolute value of the weighting factor is set low for target miRNAs which have a low relevance.

The relevance between each target miRNA and morbidity of pancreatic cancer may be estimated by a binomial or multinomial logistic regression model. Estimation methods in the binomial or multinomial logistic regression model include regularization estimation (Ridge, etc.) and sparse estimation (Lasso, SCAD, MCP, etc.). Not limited to binomial or multinomial logistic regression models, other estimation methods, such as statistical methods (e.g. ANOVA analysis or Kruskal-Wallis analysis, etc.) or machine learning methods, may be used to estimate the relevance between each target miRNA and morbidity of pancreatic cancer.

In the determination algorithm construction step (S23), a criterion value regarding pancreatic cancer incidence is also determined. For example, the determination score calculated for the pancreatic cancer-affected patient among the samples for training and the determination score calculated for the pancreatic cancer-unaffected individual among the samples for training may be referred to, and the threshold value of the determination score which can significantly discriminate both may be determined as the criterion. Whether the discrimination between the two is significant or not can be determined by whether the probability value (e.g., sensitivity, specificity, etc.) regarding the identification performance, which can be calculated by a statistical process, meets a certain level.

The threshold value of the determination score may be set in advance for each test subject. For example, if the determination score is calculated from the quantitative value of the target miRNA in a healthy state of the subject at an occasion such as a regular health checkup, the determination score in a healthy state can be used as the threshold value. Furthermore, when the determination score obtained from the subject after a certain period of time from the healthy state is higher or lower than the threshold value, an alarm can be issued to indicate that the subject may be suffering from pancreatic cancer. The threshold value can be different for each individual.

Furthermore, in the determination step (S13) of the method of the first embodiment, the determination score of the test subject may be calculated by applying the quantification results obtained in the quantification step (S12) to the determination algorithm constructed in the construction step (S23) (calculation step (S31)), and by comparing the determination score of the subject with a threshold value (comparison step ((S32)) to determine whether or not the subject has pancreatic cancer. In the comparison step (S32), the probability that the subject has pancreatic cancer may be calculated according to the size of the difference between the determination score of the subject and the threshold value. For example, it may be determined that when the difference between the determination score of the subject and the threshold value becomes larger, the probability that the subject has pancreatic cancer becomes higher.

According to the analysis method of the first embodiment described above, it is possible to easily determine the presence or absence of pancreatic cancer in a test subject by quantifying corrective miRNA and three or more target miRNAs in a sample derived from the test subject and comparing the obtained determination score with a criterion. In other words, the method can easily discriminate between individuals affected with pancreatic cancer and those without pancreatic cancer.

Since the present method can use serum or

plasma, which can be easily collected during medical examinations, etc., pancreatic cancer can be detected at an early stage. By using serum or plasma, etc., the physical and economic burden on the test subject can be greatly reduced compared to cytological diagnosis, etc., and the procedure is easy and less burdensome for the examiner. In addition, serum or plasma can be used for more accurate testing because the concentration of miRNAs contained therein are stable.

According to a further embodiment, determining that a test subject has pancreatic cancer also includes determining prognosis or recurrence of pancreatic cancer in the test subject. For example, as in part (c) of FIG. 1, the analysis method includes, after the quantification step (S12), a prognosis/recurrence determination step (S14) to determine, from the results of the quantification, whether there is a prognosis or recurrence of pancreatic cancer in the test subject. In the prognosis/recurrence determination step (S14), it is possible to determine, for example, that the prognosis of the pancreatic cancer in the test subject is poor, or that the pancreatic cancer has recurred or is highly likely to recur, by comparing the determination score with a threshold value. In particular, it may be desirable to use a threshold value determined for each test subject

After the determination step (S13) and/or the prognosis recurrence determination step (S14), it is also possible to select and assist in selecting the type of treatment or type of drug to be applied to the test subject according to the determination results. For example, as in part (d) of FIG. 1, the analysis method includes, after the determination step (S13) and/or the prognosis recurrence determination step (S14), a selection step (S15) to select a type of treatment method or a type of drug to be applied to the test subject according to the determination results. Here, the treatment method or drug is for the treatment of pancreatic cancer. The type of treatment or drug includes the amount, timing or duration of use of the treatment or drug.

According to a further embodiment, there is also provided an analysis method for assisting in determining whether or not a test subject has pancreatic cancer, comprising quantifying a corrective miRNA and three or more target miRNAs in a sample derived from the test subject (quantification step (S12)). The “assisting” determination includes, for example, obtaining information on the possibility that the test subject is suffering from pancreatic cancer. The “information” is, for example, information about the analysis results, which can be, for example, a determination score. According to the method, more accurate information can be obtained for determining whether the test subject has pancreatic cancer, determining a prognosis, determining whether the cancer has recurred, or selecting a treatment or drug to be applied to the test subject.

According to a further embodiment, the present analysis method can also be used for detecting pancreatic cancer cells in a sample which is not derived from the test subject. For example, when pancreatic cancer cells are artificially produced, this method can be used to confirm whether or not the cells are present in the produced cell-containing solution.

### (Marker)

According to the first embodiment, there is provided a marker for detecting pancreatic cancer of a test subject, including three or more target miRNAs.

Here, “marker” refers to a substance whose presence or absence or concentration in a sample can be detected to determine whether the sample and the test subject from which the sample is derived are in a specific state or not.

The marker for detecting pancreatic cancer of

the first embodiment can be used, for example, to determine whether or not the subject has pancreatic cancer, to determine whether or not the subject has a prognosis or recurrence, or to select a treatment or drug to be applied to the subject, by measuring its abundance (quantification value) in the sample derived from the test subject, as described above.

### (Kit)

According to the first embodiment, a kit for detecting pancreatic cancer is provided.

The kit contains nucleic acids which can bind specifically to each of target miRNAs (i.e., hybridize with the target miRNA) wherein the target miRNAs are selected from a group consisting of hsa-miR-205-5p, hsa-miR-223-5p, hsa-miR-29c-3p, hsa-miR-324-3p, hsa-miR-34a-5p, hsa-miR-483-5p, and hsa-miR-885-5p, and nucleic acids which can bind specifically to the corrective miRNA. That is, the kit may include nucleic acids specifically capable of binding to each of the target miRNAs of any one of the target miRNA combinations No. 1 to 85 in Table 2-1 to Table 2-3, and nucleic acids specifically capable of binding to the corrective miRNAs. As will be explained in the examples below, of the combinations No. 1 to 85, the target miRNA combinations of combinations No. 1 to 3, 31 to 40, and 65 to 73 show particularly excellent pancreatic cancer identification performance.

Therefore, the kit may contain nucleic acids which are specifically bindable to each of the combinations of target miRNAs of combination No. 1 to 3, 31 to 40 and 65 to 73, as well as nucleic acids which are specifically bindable to the corrective miRNAs. The kit may further include reagents which can be used in general methods for quantifying RNA, especially short stranded RNA, such as miRNA.

When the qPCR method is used to detect each miRNA, the nucleic acid which can bind specifically to each miRNA may be a reverse transcription (RT) primer for reverse transcription of each miRNA, an elongation (EL) primer for elongation of the miRNA, or an amplification primer set for amplifying each miRNA. Furthermore, it may also contain a probe nucleic acid to detect each miRNA.

RT primers are primers for obtaining the cDNA of each miRNA. RT primers contain sequences complementary to at least a part of each miRNA or sequences added to each miRNA. RT primers may further contain artificial sequences which are added to the CDNA to facilitate amplification of the cDNA of each miRNA.

EL primers are primers for adding artificial sequences to the cDNA to facilitate amplification of the cDNA of each miRNA. EL primers can include a sequence complementary to at least a part of the cDNA of each miRNA and an additional sequence for extension of each CDNA. An adapter sequence may also be added to each cDNA to facilitate amplification of each cDNA.

The primer set for amplification includes at least a forward primer and a reverse primer, for example, if it is for the PCR method. Alternatively, the amplification primer set may be used for the LAMP method. The primer set may include primers of sequences corresponding to each miRNA sequence used in the general LAMP method. Alternatively, the primer set may be for the NGS method, for example, and may include a forward primer containing an artificial adapter sequence and a reverse primer containing its complementary sequence. The amplification primer set for the NGS method may include multiple combinations of forward and reverse primers containing different barcode sequences for simultaneous analysis of multiple specimens. When the amplification primer set is used for rolling cycle amplification methods, the kit further includes cyclic single-stranded DNA that the amplification primers hybridize to and serve as a template for amplification.

Each primer in the amplification primer set may be designed to bind to the cDNA of each miRNA or its complementary sequence, or to bind to the artificial sequence added by the RT primer, EL primer and/or adapter sequence.

Furthermore, when each miRNA in the sample is directly detected by the microarray, the nucleic acid which can bind specifically to each miRNA is a nucleic acid probe provided by the microarray. The nucleic acid probe can have the sequence of at least a part of each miRNA, its cDNA, or its amplification product, or its complementary sequence.

The above nucleic acids in the kit may be provided individually or any of them may be combined and contained in a container with a suitable carrier. Suitable carriers are, for example, water, physiological solutions or buffers. Alternatively, it may be provided in a dry state. The container is, for example, a tube or microtiter plate. Alternatively, these nucleic acids may be provided immobilized on a solid phase such as a microfluidic chip.

The above nucleic acids in the kit may contain modified nucleic acids such as LNA, BNA, methylated nucleic acids such as 2′-OMe, and cross-linked nucleic acids. They may also be nucleic acids modified with molecules such as MGB, FITC, DIG, biotin, etc. It may also be labeled with substances used to detect optical signals, such as fluorescent substances such as FAM, ROX, JOE, VIC, HEX, Cy3, Cy5, AlexaFluor, ATTO, IRDye, and quenching substances such as TAMRA, Iowa Black, BHQ, Zen, Eclipse.

In addition to the above nucleic acids, the kit may contain reagents used for reverse transcription, elongation or amplification, such as enzymes, substrates and/or labeling substances which produce optical or electrochemical signals for detection. The labeling substances are, for example, fluorescent dyes such as SYBR Green, EvaGreen (registered trademark), SYTO (registered trademark) 82, or in the case of current detection, indicators such as metal complexes such as ruthenium hexamine.

The kit can be used, for example, to determine whether or not a patient has pancreatic cancer, to determine prognosis, to determine whether or not the disease has recurred, and to select the type of treatment or drug in the test subject as described above.

According to a further embodiment, the kit for detecting pancreatic cancer is provided as a composition for diagnosing pancreatic cancer or a diagnostic agent. Also according to an embodiment, the use of the above nucleic acid in the production of a composition for diagnosis of pancreatic cancer or a diagnostic agent for pancreatic cancer is also provided.

### EXAMPLES

Hereinafter, the experiments conducted to obtain the markers and the verification using the determination algorithm constructed with the markers will be described.

### Example 1. Comprehensive Search for Pancreatic Cancer Affected Subject Markers

Some miRNA markers which can discriminate pancreatic cancer-affected subjects from healthy subjects and non-pancreatic cancer-affected subjects were searched as follows.

### Specimen Preparation

Serum from breast cancer patients, pancreatic cancer patients, lung cancer patients, colorectal cancer patients, and healthy subjects were prepared for approximately 1000 samples (see Table 3). For the purpose of creating the determination model described below, the specimen groups were randomly assigned to two groups, Group A (approximately 600 specimens) and Group B (approximately 400 specimens).

TABLE 3

| Cancer | | | |
|---|---|---|---|
| type | Group A | Group B | Sum |
| Non-cancer | 81 | 62 | 143 |
| Colorectal | 95 | 67 | 162 |
| cancer | | | |
| Pancreatic | 82 | 66 | 148 |
| cancer | | | |
| Breast | 99 | 69 | 168 |
| cancer | | | |
| Stomach | 97 | 64 | 161 |
| cancer | | | |
| Lung | 98 | 63 | 161 |
| cancer | | | |
| Sum | 552 | 391 | 943 |

Sample Processing and miRNA Quantification

miRNA was extracted from 300 μL of serum using Nucleospin (registered trademark) miRNA Plasma (manufactured by Macherey Nagel incorporated).

The TaqMan miRNA CDNA Synthesis Kit (Applied Biosystems, catalog A28007) was used for cDNA synthesis. TaqMan miRNA CDNA Synthesis Kit is a kit which performs addition of poly (A) chain to the 3′ end of the mature RNA and ligation of an adapter sequence to the 5′ end for not target-specific but general reverse transcription of all mature RNA present in the sample. Thus, CDNAs corresponding to all mature RNAs, including miRNAs, in each sample were obtained.

The TaqMan PCR method is characterized by the combination of a primer to amplify a target and a TaqMan probe to specifically bind to the target. The probe contains fluorescent substrates that undergo fluorescence resonance energy transfer (FRET), and 5′ exonuclease activity cleaves the probe during amplification, resulting in an increase in fluorescence. This method allows accurate quantification of DNA. The concentrations of a target miRNA in the samples was determined from a calibration curve generated with known concentrations of the miRNA. The calibration curve was generated using the number of cycles until the signal obtained by amplification of the target cDNA reached the threshold (Ct value), and its concentration. For samples of unknown concentration, the concentration was determined from the Ct value.

Note that, the quantification value of each miRNA was corrected using the quantification value of hsa-miR-486-5p (SEQ ID No. 8), which is a corrective miRNA, to obtain the expression level ratio.

### Construction of Determination Algorithm

The expression level ratios obtained for each of Group A and Group B were used to construct a determination algorithm as follows.

Specifically, a determination algorithm A for pancreatic cancer was constructed by providing the quantification data obtained in Group A as a training data, and the algorithm A was validated using the quantitative data obtained in Group B as test data. Furthermore, a determination algorithm B was constructed by providing the quantitative value data obtained in Group B as a training data, and the quantification value obtained in Group A was used as test data to validate the algorithm B. In this manner, by splitting into the sample groups, training models from each sample group, and validating models against each other as test data, it is possible to extract and select combinations of miRNAs which function as pancreatic cancer markers with higher robustness.

Extraction of miRNA Combinations and Results of Verification of Identification Performance

Area Under the Curve (AUC) values of Receiver Operating Characteristic (ROC) curves were calculated for all combinations of miRNAs, which could be detected and quantified, using the established pancreatic cancer determination algorithms. An AUC value close to 1.0indicates a higher accuracy in detecting pancreatic cancer. The AUC value of 0.73 or higher was used to select 85 combinations of miRNAs, as shown in Table 4-1 to Table 4-3 below. The AUC values listed in Tables 4-1 to 4-3 are the average of the AUC values calculated for each determination algorithm A and B.

TABLE 4

| Combination | miRNA | miRNA | miRNA | miRNA | miRNA | |
|---|---|---|---|---|---|---|
| No. | 1 | 2 | 3 | 4 | 5 | AUC |
| 1 | 205-5p | 324-3p | 483-5p | | | 0.79 |
| 2 | 205-5p | 223-5p | 483-5p | | | 0.79 |
| 3 | 205-5p | 29c-3p | 483-5p | | | 0.79 |
| 4 | 29c-3p | 324-3p | 483-5p | | | 0.78 |
| 5 | 223-5p | 324-3p | 483-5p | | | 0.78 |
| 6 | 324-3p | 34a-5p | 483-5p | | | 0.78 |
| 7 | 223-5p | 29c-3p | 483-5p | | | 0.78 |
| 8 | 29c-3p | 483-5p | 885-5p | | | 0.77 |
| 9 | 29c-3p | 34a-5p | 483-5p | | | 0.77 |
| 10 | 205-5p | 29c-3p | 885-5p | | | 0.77 |
| 11 | 324-3p | 483-5p | 885-5p | | | 0.77 |
| 12 | 29c-3p | 324-3p | 885-5p | | | 0.77 |
| 13 | 29c-3p | 324-3p | 34a-5p | | | 0.77 |
| 14 | 205-5p | 324-3p | 885-5p | | | 0.77 |
| 15 | 223-5p | 483-5p | 885-5p | | | 0.77 |
| 16 | 205-5p | 223-5p | 885-5p | | | 0.76 |
| 17 | 223-5p | 29c-3p | 885-5p | | | 0.76 |
| 18 | 205-5p | 29c-3p | 34a-5p | | | 0.76 |
| 19 | 205-5p | 483-5p | 885-5p | | | 0.76 |
| 20 | 205-5p | 324-3p | 34a-5p | | | 0.76 |
| 21 | 223-5p | 324-3p | 885-5p | | | 0.76 |
| 22 | 29c-3p | 34a-5p | 885-5p | | | 0.76 |
| 23 | 223-5p | 29c-3p | 34a-5p | | | 0.75 |
| 24 | 223-5p | 324-3p | 34a-5p | | | 0.75 |
| 25 | 205-5p | 223-5p | 34a-5p | | | 0.75 |
| 26 | 223-5p | 34a-5p | 483-5p | | | 0.75 |
| 27 | 324-3p | 34a-5p | 885-5p | | | 0.75 |
| 28 | 205-5p | 34a-5p | 483-5p | | | 0.75 |
| 29 | 223-5p | 34a-5p | 885-5p | | | 0.73 |
| 30 | 205-5p | 34a-5p | 885-5p | | | 0.73 |
| 31 | 29c-3p | 324-3p | 34a-5p | 483-5p | | 0.79 |
| 32 | 205-5p | 29c-3p | 324-3p | 483-5p | | 0.79 |
| 33 | 205-5p | 324-3p | 34a-5p | 483-5p | | 0.79 |
| 34 | 205-5p | 29c-3p | 34a-5p | 483-5p | | 0.79 |
| 35 | 205-5p | 223-5p | 324-3p | 483-5p | | 0.79 |
| 36 | 205-5p | 223-5p | 29c-3p | 483-5p | | 0.79 |
| 37 | 29c-3p | 324-3p | 483-5p | 885-5p | | 0.79 |
| 38 | 205-5p | 29c-3p | 483-5p | 885-5p | | 0.79 |
| 39 | 223-5p | 324-3p | 34a-5p | 483-5p | | 0.79 |
| 40 | 205-5p | 324-3p | 483-5p | 885-5p | | 0.79 |
| 41 | 205-5p | 223-5p | 483-5p | 885-5p | | 0.78 |
| 42 | 223-5p | 29c-3p | 324-3p | 483-5p | | 0.78 |
| 43 | 223-5p | 29c-3p | 483-5p | 885-5p | | 0.78 |
| 44 | 223-5p | 29c-3p | 34a-5p | 483-5p | | 0.78 |
| 45 | 223-5p | 324-3p | 483-5p | 885-5p | | 0.78 |
| 46 | 205-5p | 223-5p | 34a-5p | 483-5p | | 0.78 |
| 47 | 205-5p | 29c-3p | 324-3p | 885-5p | | 0.77 |
| 48 | 205-5p | 29c-3p | 324-3p | 34a-5p | | 0.77 |
| 49 | 29c-3p | 324-3p | 34a-5p | 885-5p | | 0.77 |
| 50 | 205-5p | 29c-3p | 34a-5p | 885-5p | | 0.77 |
| 51 | 223-5p | 29c-3p | 324-3p | 34a-5p | | 0.77 |
| 52 | 205-5p | 223-5p | 29c-3p | 885-5p | | 0.77 |
| 53 | 205-5p | 223-5p | 324-3p | 885-5p | | 0.77 |
| 54 | 223-5p | 29c-3p | 324-3p | 885-5p | | 0.77 |
| 55 | 205-5p | 223-5p | 29c-3p | 34a-5p | | 0.77 |
| 56 | 324-3p | 34a-5p | 483-5p | 885-5p | | 0.76 |
| 57 | 205-5p | 324-3p | 34a-5p | 885-5p | | 0.76 |
| 58 | 223-5p | 29c-3p | 34a-5p | 885-5p | | 0.76 |
| 59 | 205-5p | 223-5p | 324-3p | 34a-5p | | 0.76 |
| 60 | 29c-3p | 34a-5p | 483-5p | 885-5p | | 0.76 |
| 61 | 223-5p | 324-3p | 34a-5p | 885-5p | | 0.76 |
| 62 | 205-5p | 223-5p | 34a-5p | 885-5p | | 0.75 |
| 63 | 223-5p | 34a-5p | 483-5p | 885-5p | | 0.74 |
| 64 | 205-5p | 34a-5p | 483-5p | 885-5p | | 0.74 |
| 65 | 205-5p | 29c-3p | 324-3p | 34a-5p | 483-5p | 0.80 |
| 66 | 205-5p | 29c-3p | 324-3p | 483-5p | 885-5p | 0.80 |
| 67 | 205-5p | 223-5p | 324-3p | 34a-5p | 483-5p | 0.80 |
| 68 | 205-5p | 223-5p | 29c-3p | 34a-5p | 483-5p | 0.80 |
| 69 | 223-5p | 29c-3p | 324-3p | 34a-5p | 483-5p | 0.79 |
| 70 | 205-5p | 223-5p | 29c-3p | 324-3p | 483-5p | 0.79 |
| 71 | 205-5p | 223-5p | 29c-3p | 483-5p | 885-5p | 0.79 |
| 72 | 205-5p | 223-5p | 324-3p | 483-5p | 885-5p | 0.79 |
| 73 | 223-5p | 29c-3p | 324-3p | 483-5p | 885-5p | 0.79 |
| 74 | 29c-3p | 324-3p | 34a-5p | 483-5p | 885-5p | 0.78 |
| 75 | 205-5p | 324-3p | 34a-5p | 483-5p | 885-5p | 0.78 |
| 65 | 205-5p | 29c-3p | 324-3p | 34a-5p | 483-5p | 0.80 |
| 66 | 205-5p | 29c-3p | 324-3p | 483-5p | 885-5p | 0.80 |
| 67 | 205-5p | 223-5p | 324-3p | 34a-5p | 483-5p | 0.80 |
| 68 | 205-5p | 223-5p | 29c-3p | 34a-5p | 483-5p | 0.80 |
| 69 | 223-5p | 29c-3p | 324-3p | 34a-5p | 483-5p | 0.79 |
| 70 | 205-5p | 223-5p | 29c-3p | 324-3p | 483-5p | 0.79 |
| 71 | 205-5p | 223-5p | 29c-3p | 483-5p | 885-5p | 0.79 |
| 72 | 205-5p | 223-5p | 324-3p | 483-5p | 885-5p | 0.79 |
| 73 | 223-5p | 29c-3p | 324-3p | 483-5p | 885-5p | 0.79 |
| 74 | 29c-3p | 324-3p | 34a-5p | 483-5p | 885-5p | 0.78 |

The miRNAs comprising all combinations of miRNAs shown in Tables 4-1 to 4-3 (No. 1 to 85) are three or more of hsa-miR-205-5p, hsa-miR-223-5p, hsa-miR-29c-3p, hsa-miR-324-3p, hsa-miR-34a-5p, hsa-miR-483-5p and hsa-miR-885-5p. Thus, a combination of three or more of hsa-miR-205-5p, hsa-miR-223-5p, hsa-miR-29c-3p, hsa-miR-324-3p, hsa-miR-34a-5p, hsa-miR-483-5p and hsa-miR-885-5p, that is, the combination of three or more of the target miRNAs listed in Table 1, can distinguish pancreatic cancer affected subjects from healthy subjects and other cancer affected subjects, indicating that the combination has excellent pancreatic cancer identification performance.

In particular, the combination of miRNAs selected from the miRNA group is more preferred if the combination consists of the following combinations with observed AUC values of 0.79 or higher: combination consisting of hsa-miR-205-5p, hsa-miR-324-3p and hsa-miR-483-5p (combination No. 1), combination consisting of hsa-miR-205-5p, hsa-miR-223-5p and hsa-miR-483-5p (combination No. 2), combination consisting of hsa-miR-205-5p, hsa-miR-29c-3p and hsa-miR-483-5p (combination No. 3), combination consisting of hsa-miR-29c-3p, hsa-miR-324-3p, hsa-miR-34a-5p, and hsa-miR-483-5p (combination No. 31), combination consisting of 483-5p hsa-miR-205-5p, hsa-miR-29c-3p, hsa-miR-324-3p and hsa-miR-483-5p (combination No. 32), combination consisting of hsa-miR-205-5p, hsa-miR-324-3p, hsa-miR-34a-5p and hsa-miR-483-5p (combination No. 33), combination consisting of hsa-miR-205-5p, hsa-miR-29c-3p, hsa-miR-34a-5p and hsa-miR-483-5p (combination No. 34), combination consisting of hsa-miR-205-5p, hsa-miR-223-5p, hsa-miR-324-3p and hsa-miR-483-5p (combination No. 35), combination consisting of hsa-miR-205-5p, hsa-miR-223-5p, hsa-miR-29c-3p and hsa-miR-483-5p (combination No. 36), combination consisting of hsa-miR-29c-3p, hsa-miR-324-3p, hsa-miR-483-5p and hsa-miR-885-5p (combination No. 37), combination consisting of hsa-miR-205-5p, hsa-miR-29c-3p, hsa-miR-483-5p and hsa-miR-885-5p (combination No. 38), combination consisting of hsa-miR-223-5p, hsa-miR-324-3p, hsa-miR-34a-5p and hsa-miR-483-5p (combination No. 39), combination consisting of hsa-miR-205-5p, hsa-miR-324-3p, hsa-miR-483-5p and hsa-miR-885-5p (combination No. 40), combination consisting of hsa-miR-205-5p, hsa-miR-29c-3p, hsa-miR-324-3p, hsa-miR-34a-5p and hsa-miR-483-5p (combination No. 65), combination consisting of hsa-miR-205-5p, hsa-miR-29c-3p, hsa-miR-324-3p, hsa-miR-483-5p and hsa-miR-885-5p (combination No. 66), combination consisting of hsa-miR-205-5p, hsa-miR-223-5p, hsa-miR-324-3p, hsa-miR-34a-5p and hsa-miR-483-5p (combination No. 67), combination consisting of hsa-miR-205-5p, hsa-miR-223-5p, hsa-miR-29c-3p, hsa-miR-34a-5p and hsa-miR-483-5p (combination No. 68), combination consisting of hsa-miR-223-5p, hsa-miR-29c-3p, hsa-miR-324-3p, hsa-miR-34a-5p and hsa-miR-483-5p (combination No. 69), combination consisting of hsa-miR-205-5p, hsa-miR-223-5p, hsa-miR-29c-3p, hsa-miR-324-3p and hsa-miR-483-5p (combination No. 70), combination consisting of hsa-miR-205-5p, hsa-miR-223-5p, hsa-miR-29c-3p, hsa-miR-483-5p and hsa-miR-885-5p (combination No. 71), combination consisting of hsa-miR-205-5p, hsa-miR-223-5p, hsa-miR-223-5p, hsa-miR-324-3p, hsa-miR-483-5p and hsa-miR-885-5p (combination No. 72), and combination consisting of hsa-miR-223-5p, hsa-miR-29c-3p, hsa-miR-324-3p, hsa-miR-483-5p and hsa-miR-885-5p (combination No. 73).

### Example 2: Identification Performance of Target miRNAs Alone and in Combination with miRNAs Other than Target miRNAs

Pancreatic Cancer Identification Performance of Targeted miRNAs Alone

As a comparative example, the identification performance of the target miRNAs listed in Table 1 (sequence numbers 1 to 7) used alone as pancreatic cancer markers was verified by determining AUC values using a determination algorithm as in Example 1.

### Verification Results

The verification results are shown in Table 5. Compared to the AUC values of combinations 1 to 85 described in Example 1, it can be seen that the target miRNA alone does not have sufficient pancreatic cancer identification performance.

TABLE 5

| SEQ ID | miRNA | |
|---|---|---|
| NO. | name | AUC |
| 1 | 205-5p | 0.62 |
| 2 | 223-5p | 0.62 |
| 3 | 29c-3p | 0.63 |
| 4 | 324-3p | 0.65 |
| 5 | 34a-5p | 0.59 |
| 6 | 483-5p | 0.63 |
| 7 | 885-5p | 0.65 |

Pancreatic Cancer Identification Performance with miRNAs Other Than Target miRNAs

As a further comparative example, the pancreatic cancer identification performance of combinations of miRNAs other than the target miRNAs was also verified. Specifically, combinations of miRNAs of SEQ ID No. 9 to 14 listed in Table 6 below were verified by obtaining AUC values using the same determination algorithm as in Example 1. Each miRNA listed in Table 6 has been known as a marker for primary screening of cancer and can discriminate between healthy subjects and populations of affected subjects with breast, pancreatic, lung, stomach, and colorectal cancer. However, it has also been known that each miRNA listed in Table 6 only reveals differences from healthy subjects as a primary screening marker for cancer and does not identify cancer types (that is, it does not identify pancreatic cancer patients, for example, from a group of samples from patients with breast, pancreatic, lung, stomach,

TABLE 6

| SEQ | | |
|---|---|---|
| ID | | |
| NO. | miRNA name | Base sequence |
| 9 | hsa-miR-106b-5p | UAAAGUGCUGACAGUGCAGAU |
| 10 | hsa-miR-125a-5p | UCCCUGAGACCCUUUAACCUGUGA |
| 11 | hsa-miR-190a-5p | UGAUAUGUUUGAUAUAUUAGGU |
| 12 | hsa-miR-30e-5p | UGUAAACAUCCUUGACUGGAAG |
| 13 | hsa-miR-3613-3p | ACAAAAAAAAAAGCCCAACCCUUC |
| 14 | hsa-miR-3613-5p | UGUUGUACUUUUUUUUUUGUUC |

### Verification Results

The validation results are shown in Table 7. In Table 7, for example, hsa-miR-106b-5p is listed with the abbreviation “106b-5p”, and other miRNAs are listed with similar abbreviations.

Compared to the AUC values of the combinations of target miRNAs of the present embodiment (No. 1 to 85) described in Example 1, the AUC values of the combinations of miRNAs (No. 86 to 91) described in Table 6 are lower. Thus, it is found that the combination of target miRNAs in the present embodiment exhibited better pancreatic cancer identification performance than the combination of miRNAs other than the target miRNAs.

TABLE 7

| Combination | miRNA | miRNA | miRNA | miRNA | miRNA | |
|---|---|---|---|---|---|---|
| No. | 1 | 2 | 3 | 4 | 5 | AUC |
| 86 | 106b-5p | 125a-5p | 30e-5p | 3613-3p | 3613-5p | 0.60 |
| 87 | 125a-5p | 190a-5p | 30e-5p | 3613-3p | 3613-5p | 0.59 |
| 88 | 106b-5p | 125a-5p | 190a-5p | 30e-5p | 3613-5p | 0.57 |
| 89 | 106b-5p | 125a-5p | 190a-5p | 3613-3p | 3613-5p | 0.56 |
| 90 | 106b-5p | 125a-5p | 190a-5p | 30e-5p | 3613-3p | 0.55 |
| 91 | 106b-5p | 190a-5p | 30e-5p | 3613-3p | 3613-5p | 0.53 |

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An analysis method for determining a presence or absence of pancreatic cancer in an test subject, comprising: quantifying corrective miRNA and target miRNAs in a sample derived from the test subject, wherein the target miRNAs are three or more selected from a group consisting of hsa-miR-205-5p, hsa-miR-223-5p, hsa-miR-29c-3p, hsa-miR-324-3p, hsa-miR-34a-5p, hsa-miR-483-5p and hsa-miR-885-5p.

2. The method of claim 1, wherein miRNAs selected from the group of miRNAs are hsa-miR-205-5p, hsa-miR-324-3p, and hsa-miR-483-5p.

3. The method of claim 1, wherein miRNAs selected from the group of miRNAs are hsa-miR-205-5p, hsa-miR-223-5p, and hsa-miR-483-5p.

4. The method of claim 1, wherein miRNAs selected from the group of miRNAs are hsa-miR-205-5p, hsa-miR-29c-3p, and hsa-miR-483-5p.

5. The method of claim 1, wherein miRNAs selected from the group of miRNAs are hsa-miR-29c-3p, hsa-miR-324-3p, hsa-miR-34a-5p, and hsa-miR-483-5p.

6. The method of claim 1, wherein miRNAs selected from the group of miRNAs are hsa-miR-205-5p, hsa-miR-29c-3p, hsa-miR-324-3p, and hsa-miR-483-5p.

7. The method of claim 1, wherein miRNAs selected from the group of miRNAs are hsa-miR-205-5p, hsa-miR-324-3p, hsa-miR-34a-5p, and hsa-miR-483-5p.

8. The method of claim 1, wherein miRNAs selected from the group of miRNAs are hsa-miR-205-5p, hsa-miR-29c-3p, hsa-miR-34a-5p, and hsa-miR-483-5p.

9. The method of claim 1, wherein miRNAs selected from the group of miRNAs are hsa-miR-205-5p, hsa-miR-223-5p, hsa-miR-324-3p, and hsa-miR-483-5p.

10. The method of claim 1, wherein miRNAs selected from the group of miRNAs are hsa-miR-205-5p, hsa-miR-223-5p, hsa-miR-29c-3p, and hsa-miR-483-5p.

11. The method of claim 1, wherein miRNAs selected from the group of miRNAs are hsa-miR-29c-3p, hsa-miR-324-3p, hsa-miR-483-5p, and hsa-miR-885-5p.

12. The method of claim 1, wherein miRNAs selected from the group of miRNAs are hsa-miR-205-5p, hsa-miR-29c-3p, hsa-miR-483-5p, and hsa-miR-885-5p.

13. The method of claim 1, wherein miRNAs selected from the group of miRNAs are hsa-miR-223-5p, hsa-miR-324-3p, hsa-miR-34a-5p, and hsa-miR-483-5p.

14. The method of claim 1, wherein miRNAs selected from the group of miRNAs are hsa-miR-205-5p, hsa-miR-324-3p, hsa-miR-483-5p, and hsa-miR-885-5p.

15. The method of claim 1, wherein miRNAs selected from the group of miRNAs are hsa-miR-205-5p, hsa-miR-29c-3p, hsa-miR-324-3p, hsa-miR-34a-5p, and hsa-miR-483-5p.

16. The method of claim 1, wherein miRNAs selected from the group of miRNAs are hsa-miR-205-5p, hsa-miR-29c-3p, hsa-miR-324-3p, hsa-miR-483-5p, and hsa-miR-885-5p.

17. The method of claim 1, wherein miRNAs selected from the group of miRNAs are hsa-miR-205-5p, hsa-miR-223-5p, hsa-miR-324-3p, hsa-miR-34a-5p, and hsa-miR-483-5p.

18. The method of claim 1, wherein miRNAs selected from the group of miRNAs are hsa-miR-205-5p, hsa-miR-223-5p, hsa-miR-29c-3p, hsa-miR-34a-5p, and hsa-miR-483-5p.

19. The method of claim 1, wherein miRNAs selected from the group of miRNAs are hsa-miR-223-5p, hsa-miR-29c-3p, hsa-miR-324-3p, hsa-miR-34a-5p, and hsa-miR-483-5p.

20. The method of claim 1, wherein miRNAs selected from the group of miRNAs are hsa-miR-205-5p, hsa-miR-223-5p, hsa-miR-29c-3p, hsa-miR-324-3p, and hsa-miR-483-5p.

21. The method of claim 1, wherein miRNAs selected from the group of miRNAs are hsa-miR-205-5p, hsa-miR-223-5p, hsa-miR-29c-3p, hsa-miR-483-5p, and hsa-miR-885-5p.

22. The method of claim 1, wherein miRNAs selected from the group of miRNAs are hsa-miR-205-5p, hsa-miR-223-5p, hsa-miR-324-3p, hsa-miR-483-5p, and hsa-miR-885-5p.

23. The method of claim 1, wherein miRNAs selected from the group of miRNAs are hsa-miR-223-5p, hsa-miR-29c-3p, hsa-miR-324-3p, hsa-miR-483-5p, and hsa-miR-885-5p.

24. The method of claim 1, wherein a quantification value of the target miRNAs is normalized by a quantification value of the corrective miRNA.

25. The method of claim 24, wherein the corrective miRNA is hsa-miR-486-5p.

26. The method of claim 1, further comprising:
(S21) preparing a sample for training derived from an individual who is known to have pancreatic cancer or not;
(S22) quantifying the corrective miRNA and the three or more target miRNAs in said sample for training; and
(S23) constructing a determination algorithm to determine whether or not the individual has pancreatic cancer based on the information about the individual, the quantification value of the corrective miRNA, and the quantification value of the three or more target miRNAs in the sample for training,
wherein whether or not the test subject has pancreatic cancer is determined by applying the quantification value of the corrective miRNA and the quantification value of the target miRNAs in the sample derived from the test subject to the determination algorithm.

27. The method of claim 26, wherein the individual is a healthy subjects or a patients known to have cancer other than pancreatic cancer.

28. The method of claim 27, wherein the patients have breast cancer, lung cancer, stomach cancer, or colorectal cancer.

29. The method of claim 26, wherein the information about the individual includes information about whether or not the individual has pancreatic cancer.

30. The method of claim 26, wherein the determination algorithm is constructed by binomial logistic regression, multinomial logistic regression, statistical methods, or modeling by machine learning.

31. The method of claim 30, wherein the binomial logistic regression and the multinomial logistic regression are regularized or sparse estimation, and said modeling by machine learning is modeling using a linear model, nonlinear model, Bayesian model, support vector machine model, random forest model, boosting model, or neural network model.

32. The method of claim 1, wherein the quantification is performed using PCR, LAMP, next generation sequencing, or microarray method.

33. The method of claim 1, wherein the sample is serum or plasma.

34. A kit configured to detect pancreatic cancer, the kit including nucleic acid which binds specifically to a target miRNAs, wherein the target miRNAs are three or more selected from a group consisting of hsa-miR-205-5p, hsa-miR-223-5p, hsa-miR-29c-3p, hsa-miR-324-3p, hsa-miR-34a-5p, hsa-miR-483-5p, and hsa-miR-885-5p, and nucleic acid which binds specifically to has-miR-486-5p.

35. The kit of claim 34, wherein the nucleic acid which bind specifically to the target miRNAs is nucleic acid for reverse transcription, nucleic acid for elongation, nucleic acid set for amplification, or nucleic acid probe or nucleic acid primer set for detection.

36. The kit of claim 34, wherein the miRNAs selected from the miRNA group are hsa-miR-205-5p, hsa-miR-324-3p, and hsa-miR-483-5p.
